(19) United States
(12) Patent Application Publication
(10) Pub. No.: US 2009/0306429 A1
(43) Pub. Date: Dec. 10, 2009

(54) PREPARATION OF A QUATERNARY AMMONIUM HYDROXIDE AND USE THEREOF FOR THE PREPARATION OF A QUATERNARY AMMONIUM SALT

(75) Inventors: Rajnikant Vyas Janmejay, London (GB); Jayshree H. Shah, Ahmedabad (IN); Ravindra Niranjanbhai Raval, Ahmedabad (IN)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignees: Henk Pluim, Hierden, NL (NL); DISHMAN PHARMACEUTICALS AND CHEMICALS LTD, Ahmedabad, IN (IN)

(21) Appl. No.: 11/720,931

(22) PCT Filed: Nov. 30, 2005
(86) PCT No.: PCT/NL05/50055

§ 371 Date: Jun. 4, 2009

(30) Foreign Application Priority Data
Dec. 6, 2004 (US) 60632976

(51) Int. Cl. C07C 237/20

(52) U.S. Cl. 564/194

(57) ABSTRACT

The invention relates to a process for the preparation of a quaternary ammonium hydroxide, in particular denatonium hydroxide, and the use thereof for the preparation of a quaternary ammonium salt, in particular of denatonium benzoate or a denatonium fatty acid derivative. Due to its extremely bitter taste, this latter compound is applied in the art as an aversive agent, biocide, antifoulant and flavorant. It is usually prepared from a quaternary ammonium halide in an organic environment, which after isolation and purification is converted to a hygroscopic and unstable hydroxide intermediate, which in turn is immediately converted to the salt of interest in the solvent in which it is prepared. It is now found that these isolation steps can be avoided by performing the reaction at aqueous conditions. This is more straightforwardly and does not require costly precautions to avoid contact while handling. Moreover, the process of the invention allows to produce the quaternary ammonium salt from a lignocaine compound in a one-pot synthesis involving mostly water.

### TECHNICAL FIELD OF THE INVENTION

The invention relates to a process for the preparation of a quaternary ammonium hydroxide, in particular N-{2-[(2,6-dimethylphenyl)-amino]-2-oxoethyl}-N,N-diethyl-benzenemethanaminium hydroxide (i.e. denatonium hydroxide), and the use thereof for the preparation of a quaternary ammonium salt, in particular of denatonium benzoate or a denatonium fatty acid derivative.

### BACKGROUND OF THE INVENTION

Quaternary ammonium compounds, in particular denatonium benzoate or denatonium capsaicinate, provide an enhanced bitter and/or spicy, pungent flavor, and for that reason may be applied as an aversive agent, biocide, antifoulant or flavorant. These compounds are often incorporated into compositions or coatings in order to deter either ingestion by humans or to repel mammals, reptiles, fish and birds from an article coated therewith, but also the use as a denaturant for alcohol and tallow or as a bitter flavoring agent in food.

It is known in the art to produce denatonium benzoate, or related quaternary salts, starting from a denatonium halide. For example, GB 955,309 teaches a synthesis route for a quaternary ammonium benzoate involving denatonium chloride, wherein the denatonium chloride is first prepared by reacting lignocaine with benzyl chloride in an organic solvent, and then contacted with alcoholic caustic alkali or an ion-exchange resin in the hydroxide form to give denatonium hydroxide. This intermediate product is converted to denatonium benzoate, or related salts, using benzoic acid and methanol.

From GB 955,309 it is generally understood that the quality of a quaternary denatonium salt such as denatonium benzoate or denatonium capsaicinate is largely determined by the purity of the starting material, i.e. the denatonium halide. In order to arrive at a quaternary ammonium salt that meets USP specifications, denatonium halide is first subjected to isolation and purification steps involving vacuum distillation in an organic environment and centrifugation or crystallization, before it is converted to a hygroscopic and unstable denatonium hydroxide intermediate. It is mentioned in GB 955,309 that the quaternary hydroxide is best used in the organic solvent in which it is prepared to produce the final quaternary ammonium compound of interest.

Because denatonium halides are known to be extremely bitter and highly contaminating compounds, every working step involving these compounds requires costly precautions to avoid contact while handling. This is especially the case where such a compound is isolated from its solvent environment.

It is therefore an object of the present invention to provide a process for the preparation of a quaternary ammonium salt, in particular denatonium benzoate or related salt, particularly a denatonium benzoate, wherein the pure quaternary ammonium salt thus prepared meets the international standards set thereto, wherein the process lacks any superfluous isolation steps, wherein the solvent conditions throughout the process do not change significantly and where possible contact with the bitter compounds used in the process is minimized.

### DESCRIPTION OF THE INVENTION

Accordingly the present invention relates to a process for the preparation of a quaternary ammonium hydroxide R⁺OH⁻, in which R⁺ has the formula:

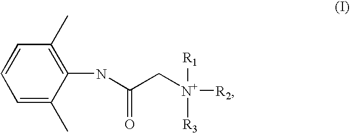

in which R₁ and R₂ each independently represent an alkyl group containing from 1 to 4 carbon atoms, for example methyl, ethyl, n-propyl or n-butyl groups, and R₃ represents a benzyl group or a substituted benzyl group, such as o- or p-chlorobenzyl, said process comprising forming an aqueous solution containing a quaternary ammonium halide having said cation R⁺, and said halide preferably being a Cl⁻ ion, contacting said aqueous solution with hydroxide ions, to obtain a quaternary ammonium hydroxide, and isolating said quaternary ammonium hydroxide.

In the process of the invention the quaternary ammonium hydroxide rather than the corresponding ammonium halide starting material is isolated. It is found that the purity of the hydroxide form has a direct effect on the quality of the final product to be prepared from this compound, but that the quaternary ammonium hydroxide is isolated much more straightforwardly than the halide form, despite the warnings for instability of the hydroxide given in GB 955,309. The invention takes advantage of the water-insolubility of the quaternary ammonium hydroxide by exchanging the halide ions with hydroxide ions in an aqueous environment rather than in the organic, mostly alcoholic environment applied in the prior art.

It is considered unnecessary and even unwanted to subject the quaternary ammonium halide after formation to any purification and isolation steps other than extraction. The extraction step allows for the removal of any starting materials or other impurities originating from the formation of the halide salt. It is considered economically favorable to provide the quaternary ammonium halide in the solvent in which it is prepared over a complete isolation of this compound using vacuum distillation and crystallization as taught in GB 955,309. Conveniently the quaternary ammonium halide is provided in an aqueous environment which is preferably washed at least once with an organic solvent, preferably toluene. Preferably the aqueous solution provided to the reaction comprises quaternary ammonium halide in an amount between 30 wt % and 90 wt %, more preferably between 50 wt % and 75 wt %, based on the weight of the aqueous solution.

The source of hydroxide ions in the above-mentioned reaction can be caustic alkali or an ion-exchange resin in the hydroxide form. It is preferred that the hydroxide ions are provided by an aqueous caustic alkali solution, preferably a sodium hydroxide solution having a concentration of at least 40 wt % and up to 60 wt %, more preferably 45-55 wt %, most preferably 48-52 wt %, based on the weight of the solution. The reaction is preferably performed at atmospheric conditions and at temperatures between 10-50° C., more preferably between 20 and 40° C., most preferably between 25 and 30° C.

The quaternary ammonium halide and the hydroxide ions are provided in such relative amounts that there is an excess hydroxide ions over quaternary ammonium halide, “excess” meaning a molar ratio of hydroxide ions to quaternary ammonium halide higher than 1:1, preferably between 1.1:1 and 2:1, more preferably between 1:1:1 and 1.3:1.

Where it is mentioned that the reaction is performed in an aqueous environment, it is preferred that the solvent comprises at least 80 wt % of water, preferably at least 90 wt %, more preferably at least 95 wt % of water, based on the total weight of the solvent, and most preferably the solvent does not contain a detectable amount of organic solvent at all. With the “solvent” it is meant both the water provided by the aqueous solution containing the quaternary ammonium halide and the solvent containing the hydroxide ions.

Purification of the quaternary ammonium hydroxide involves washing with excess water to remove salts such as sodium or potassium hydroxide present in solution. Due to its aforementioned water-insolubility, the quaternary ammonium hydroxide precipitates as it forms, and the solid can be isolated in crystalline form by centrifugation and/or filtering techniques known to those skilled in the art.

The invention also relates to a denatonium hydroxide in its crystalline form, having a melting point between 150 and 160° C., more preferably 150-157° C., preferably having an off-white appearance, which can be obtained by the above-described process.

The quaternary ammonium halide is preferably formed from the reaction of a tertiary amine of the formula:

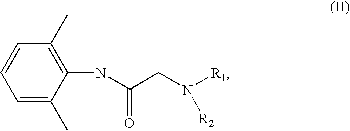

with a halide having the general formula R₃halide, wherein R₁, R₂ and R₃ each have the aforementioned meaning, and the halide being a F, Cl, Br, I group, preferably a Cl group, wherein the reaction is performed in aqueous conditions. It is preferred that the tertiary amine according to formula (II) is a lignocaine (ω-diethylamino-2,6-dimethylacetanilide or lidocaine) compound, or a derivative thereof. The lignocaine is understood to comprise both its acidic and its free base form. When the R₃-halide is highly reactive, the quaternising reaction may take place at room temperature. In most cases, however, it is necessary to employ heating, preferably involving a temperature between 50 and 100° C., more preferably 70-90° C. These conditions can readily be determined by those skilled in the art. As mentioned above, recovery of the contaminating quaternary ammonium halide is to be avoided. The halide salt thus obtained is washed at least once with an organic solvent, preferably toluene, to remove excess R₃-halide and organic impurities.

In a preferred embodiment of the present invention the lignocaine compound or derivate thereof is brought into contact with a benzyl halide, preferably a benzyl chloride, which upon reacting yields denatonium chloride (R₁, R₂=ethyl; R₃=benzyl in formula (I)). Lignocaine, having the formula (II) in which R₁ and R₂ are ethyl groups, can for instance be prepared by reacting N-chloroacetyl-2,6-dimethylaniline with diethylamine in the presence of aqueous sodium carbonate. The preferred quaternary ammonium hydroxide of the present invention is a N-{2-[(2,6-dimethylphenyl)-amino]-2-oxoethyl}-N,N-diethyl-benzenemethanaminium hydroxide or denatonium hydroxide, meaning that R₁ and R₂ are ethyl groups and R₃ is a benzyl group in formula (I).

The process of the present invention allows for the production of an extremely bitter quaternary ammonium salt from a lignocaine compound in a one-pot synthesis involving mostly water, without the necessity to intervene in between through isolation of an intermediate compound, up to the point where quaternary ammonium hydroxide is obtained as a stable solid. Extensive contact with contaminating intermediate states and distillations at reduced pressure and high temperatures can thus be avoided.

The invention also relates to a process for the preparation of a quaternary ammonium salt having a benzoate, a hydrogen phthalate, a hydrogen tartrate, a hydrogen oxalate or a capsaicinate anion, or their substituted analogues, or a fatty acid derivative, by reacting the corresponding isolated quaternary ammonium hydroxide of the invention with the conjugated acid of the anion, i.e. benzoic acid, phthalic acid, tartaric acid, oxalic acid, capsaicin, or with the fatty acid, respectively, in the presence of an organic solvent such as methanol or acetone, preferably acetone. With a fatty acid derivative it is meant the conjugated base of a fatty acid, such as palmitate, linolate, linolenate or eruciate. Fatty acids in the context of the invention are aliphatic monocarboxylic acids containing a long hydrocarbon chain, either saturated or unsaturated, preferably having a carbon chain length from C12 to C22. With the process of the present invention it is now possible to produce such quaternary ammonium salts with up to 50% production time reduction.

The quaternary ammonium salt is preferably a denatonium benzoate, prepared from the reaction of a denatonium hydroxide according to the invention with benzoic acid, preferably using acetone as the solvent.

In another embodiment the quaternary ammonium salt is preferably a denatonium fatty acid derivative, prepared from the reaction of a denatonium hydroxide with a fatty acid. The denatonium fatty acid derivative is preferably denatonium palmitate, linolate, linoleate or ericuiate.

### Example 1

### Preparation of N-{2-[(2,6-dimethylphenyl)-amino]-2-oxoethyl}-N,N-diethyl-benzenemethanaminium hydroxide (denatonium hydroxide)

250 gram (1.07 mol) of 2-(diethylamino)-N-(2,6-dimethylphenyl)acetamide (Lignocaine) was added to 600 ml of water at 30-35° C. It was then slowly heated to 70-90° C., followed by the addition of 175.4 gram (1.39 mol) benzyl chloride at 70-90° C. The temperature was maintained at 70-90° C. for 20-24 hours while monitoring the reaction by thin layer chromatography for every 4 hours, until the remaining unreacted Lignocaine was less than 10%. The reaction mass was then cooled to 35-40° C. The aqueous reaction solution was extracted with 100 ml of toluene (twice) at 35-40° C. Approximately 920 gram of aqueous denatonium chloride solution was obtained. The assay was 50.7% as measured by titrimetry.

50% sodium hydroxide solution (129.3 gram sodium hydroxide in 129.3 ml water) was added hereto, i.e. 1.25 mol sodium hydroxide, against 1 mol denatonium chloride, at 25-30° C. over a period of 3.0 hrs. The solid denatonium hydroxide was filtered and washed with 400 ml demineralised water and finally with 50 ml acetone.

After drying the residual product weighed 255 gram (Assay 98.75% by titrimetry, melting range 151-155° C., off-white crystals; LOD<1%; chloride content 0.01%, ash content 0.10%). The overall yield was 69.0% based on lignocaine.

### Example 2

### Preparation of N-{2-[(2,6-dimethylphenyl)-amino]-2-oxoethyl}-N,N-diethyl-benzenemethanaminium hydroxide (denatonium hydroxide) from N-chloroacetyl-2,6-dimethylaniline

1000 gram (5.06 mol) of N-chloroacetyl-2,6-dimethylaniline was added to 3250 ml water at 30-35° C. 280 gram of sodium carbonate and 550 gram (7.52 mol) of diethylamine was added hereto at 30-35° C. and the mixture was stirred for 2 hrs. It was then slowly heated to 60-62° C. and stirred for another 2 hrs at the same temperature. The temperature was raised to 70-90° C. and the mixture was stirred for 8 hrs at the same temperature. Then slowly 834 gram (6.59 mol) of benzyl chloride was added at 70-90° C. The temperature was maintained for 20-24 hrs at 70-90° C. while monitoring the reaction by thin layer chromatography for every 4 hrs, until the unreacted lignocaine was less than 10%. The reaction mass was cooled to 35-40° C. The aqueous reaction solution was extracted with 250 ml of toluene [twice] at 35-40° C. and the two layers separated. The upper layer was the toluene layer and the lower layer was the aqueous layer containing the denatonium chloride. About 3000 gram of an aqueous denatonium chloride solution was obtained, with a yield of 73.4% denatonium chloride, as measured by titrimetry.

50% sodium hydroxide solution (612 gram sodium hydroxide in 612 ml water) was added to the prepared denatonium chloride solution at 25-30° C. over a period of 3.0 hrs, i.e. 1.25 mol sodium hydroxide against 1 mol denatonium chloride. The isolated solid denatonium hydroxide was filtered and washed with 3000 ml of water, and finally washed with 500 ml of acetone.

After drying the product weighed 1225 gram (Assay 99% by titrimetry; melting range 152-156° C., off-white crystals; LOD<1%; chloride content 0.01%, ash content 0.10%). The overall yield was 70.7% based on N-chloroacetyl-2,6-dimethylaniline.

### Example 3

### Preparation of Preparation of Pure Quaternary Salts from Pure Quaternary Hydroxide

100 gram (0.29 mol) of denatonium hydroxide was added to 150 ml of acetone at 30-35° C. A solution of 38.91 gram (0.319 mol) of benzoic acid in 150 ml of acetone was added at 30-35° C., i.e. 1.1 mol benzoic acid against 1 mol denatonium hydroxide, and the thus obtained mixture was stirred for 30 min. at this temperature and 2.0 hrs at 30-35° C. Then the reaction mass was cooled to 18-22° C. and stirred for another 30 minutes, the solid filtered and subsequently washed with 25 ml acetone. 105 gram of denatonium benzoate was obtained on drying, yield was 80.52%. The quality met the USP specifications.

### Example 4

### Preparation of Fatty Derivative Salts from Pure Quaternary Hydroxide (Denatonium Palmitate)

50 grams (0.1462 mol) of denatonium hydroxide was dissolved in 50 ml of methanol at 30-45° C. A solution of 37.5 gram (0.1462 mol i.e. 1 mol palmitic acid against 1 mol denatonium hydroxide) of palmitic acid in 150 ml of methanol was added at 30-45° C., and the thus obtained mixture was stirred for 1-2 hours at 45-60° C. temperature. The solvent was then distilled off up to semi solid to solid mass. 84 grams of denatonium palmitate (with a quantitative yield) was obtained as off white solid, which is hygroscopic in nature. (Assay=99.41%, S′ Ash=0.15%).

### Example 5

### Preparation of Fatty Derivative Salts from Pure Quaternary Hydroxide (Denatonium Linolate)

50 grams (0.1462 mol) of denatonium hydroxide was dissolved in 50 ml of methanol at 30-45° C. A solution of 41 gram (0.1462 mol i.e. 1 mole linoleic acid against 1 mol denatonium hydroxide) of linoleic acid in 50 ml of methanol was added at 30-45° C., and the thus obtained mixture was stirred for 1-2 hours at 45-60° C. temperature. The solvent was then distilled off up to thick brownish semi solid mass. 90 grams of denatonium linolate was obtained with a quantitative yield. (Assay=100%, S′ Ash=0.09%).

### Example 6

### Preparation of Fatty Derivative Salts from Pure Quaternary Hydroxide (Denatonium Linolenate)

50 grams (0.1462 mol) of denatonium hydroxide was dissolved in 50 ml of methanol at 30-45° C. A solution of 40.7 gram (0.1462 mol i.e. 1 mole linolenic acid against 1 mol denatonium hydroxide) of linolenic acid in 50 ml of methanol was added at 30-45° C., and the thus obtained mixture was stirred for 1-2 hours at 45-60° C. temperature. The solvent was then distilled off up to thick brownish semi solid mass. 93 grams of denatonium linolenate was obtained with a quantitative yield. (Assay=99.11%, S′ Ash 0.09%).

### Example 7

### Preparation of Fatty Derivative Salts from Pure Quaternary Hydroxide (Denatonium Eruciate)

50 grams (0.1462 mol) of denatonium hydroxide was dissolved in 50 ml of methanol at 30-45° C. A solution of 54.5 gram (0.1462 mol i.e. 1 mole erucic acid against 1 mol denatonium hydroxide) of erucic acid (with 90.7% assay) in 50 ml of methanol was added at 30-45° C., and the thus obtained mixture was stirred for 1-2 hours at 45-60° C. temperature. The solvent was then distilled off up to yellow thick semi solid mass. 101 grams of denatonium eruciate was obtained with a quantitative yield. (Assay=99.09%, S′ Ash=0.08%).

1. A process for the preparation of a quaternary ammonium hydroxide R⁺OH⁻, in which R⁺ has the formula: 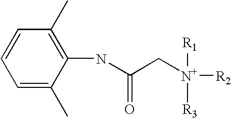
in which R₁ and R₂ each independently represent an alkyl group containing from 1 to 4 carbon atoms and R₃ represents a benzyl group or a substituted benzyl group, said process comprising forming an aqueous solution containing a quaternary ammonium halide having said cation R⁺, contacting said aqueous solution with hydroxide ions, to obtain a quaternary ammonium hydroxide, and isolating said quaternary ammonium hydroxide.

2. The process according to claim 1, wherein said quaternary ammonium halide is prepared from a lignocaine compound or derivative thereof.

3. The process according to claim 1, wherein said quaternary ammonium halide is a denatonium halide (R₁, R₂=ethyl; R₃=benzyl).

4. The process according to claim 1, wherein said quaternary ammonium hydroxide is denatonium hydroxide (R₁, R₂=ethyl; R₃=benzyl).

5. A compound, denatonium hydroxide, in its crystalline form, having a melting point between 150 and 160° C.

6. A process for the preparation of a quaternary ammonium salt having a benzoate, a hydrogen phthalate, a hydrogen tartrate or a hydrogen oxalate anion, or a fatty acid derivative, or their substituted analogues, wherein the process comprises reacting a quaternary ammonium hydroxide isolatable by the process according to claim 1 with the conjugated acid of said anion or with the fatty acid, in the presence of an organic solvent.

7. The process according to claim 6, wherein the quaternary ammonium salt is denatonium benzoate (R₁, R₂=ethyl; R₃=benzyl) or a denatonium fatty acid derivative (R₁, R₂=ethyl; R₃=fatty acid).

8. The process according to claim 2, wherein said quaternary ammonium halide is a denatonium halide (R₁, R₂=ethyl; R₃=benzyl).

9. A process for the preparation of a quaternary ammonium salt having a benzoate, a hydrogen phthalate, a hydrogen tartrate or a hydrogen oxalate anion, or a fatty acid derivative, or their substituted analogues, wherein the process comprises reacting a quaternary ammonium hydroxide isolatable by the process according to claim 2 with the conjugated acid of said anion or with the fatty acid, in the presence of an organic solvent.

10. A process for the preparation of a quaternary ammonium salt having a benzoate, a hydrogen phthalate, a hydrogen tartrate or a hydrogen oxalate anion, or a fatty acid derivative, or their substituted analogues, wherein the process comprises reacting a quaternary ammonium hydroxide isolatable by the process according to claim 3 with the conjugated acid of said anion or with the fatty acid, in the presence of an organic solvent.

11. A process for the preparation of a quaternary ammonium salt having a benzoate, a hydrogen phthalate, a hydrogen tartrate or a hydrogen oxalate anion, or a fatty acid derivative, or their substituted analogues, wherein the process comprises reacting a quaternary ammonium hydroxide isolatable by the process according to claim 4 with the conjugated acid of said anion or with the fatty acid, in the presence of an organic solvent.

12. A process for the preparation of a quaternary ammonium salt having a benzoate, a hydrogen phthalate, a hydrogen tartrate or a hydrogen oxalate anion, or a fatty acid derivative, or their substituted analogues, wherein the process comprises reacting a quaternary ammonium hydroxide isolatable by the process according to claim 8 with the conjugated acid of said anion or with the fatty acid, in the presence of an organic solvent.
